(19) United States
(12) Patent Application Publication
(10) Pub. No.: US 2002/0041888 A1
(43) Pub. Date: Apr. 11, 2002

(54) Method and composition for treating cancer by administration of apoptosis-inducing chemotherapeutic agents

(76) Inventor: Moshe Flashner-Barak, Petach Tikva (IL)

Correspondence Address: KENYON & KENYON, ONE BROADWAY, NEW YORK, NY, 10004, US

(21) Appl. No.: 09/829,621

(22) Filed: Apr. 10, 2001

Related U.S. Application Data
(60) Provisional application No. 60/195,920, filed on Apr. 10, 2000.

(51) Int. Cl. A61K 9/00

(52) U.S. Cl. 424/400

(57) ABSTRACT

The present invention provides an anti-tumor chemotherapeutic to a patient having a tumor, the composition comprising; microspheres incorporating the anti-tumor chemotherapeutic; and, a suspending solution which surrounds the microspheres. The present invention also provides a method for administering an anti-tumor chemotherapeutic to a patient having a tumor, comprising the steps of delivering the anti-tumor chemotherapeutic as a chemotherapeutic reservoir to the tumor; and, releasing the anti-tumor chemotherapeutic from the chemotherapeutic reservoir to an interstitial space of the tumor in a therapeutically effective amount, wherein, the chemotherapeutic reservoir includes microspheres incorporating the anti-tumor chemotherapeutic and a suspending solution which surrounds the microspheres.

### CROSS-REFERENCE TO RELATED APPLICATION

This application claims the benefit of provisional application Serial No. 60/195,920, filed Apr. 10, 2000, which is incorporated entirely herein by reference.

### FIELD OF THE INVENTION

The present invention relates to the field of delivery of anti-tumor chemotherapeutics.

### BACKGROUND

Paclitaxel is a high molecular weight (854g/mole), highly lipophilic cytotoxic chemotherapeutic used as an anti-tumor agent in the treatment of carcinomas of the ovary, breast, lung and in the treatment AIDS related Karposi's sarcoma. Paclitaxel is currently used to treat breast cancer by pre-operatively administering the chemotherapeutic systemically. The pre-operation treatment reduces tumor burden prior to surgery, thus potentially improving the post-surgery prognosis. Although impressive success has been achieved using this approach, the treatment requires prolonged hospitalization and is accompanied by severe side-effects. Moreover, a significant number of cases (30%) do not result in a clinically satisfactory outcome either because the tumors are not reduced or because the side effects require that paclitaxel dosing be discontinued.

Several pharmaceutical companies and research laboratories have been involved in the development of more sustained formulations of the potent chemotherapeutic agent, paclitaxel. Reservoir vehicles utilizing polymers containing microspheres of paclitaxel or gels of paclitaxel are currently undergoing clinical investigation to determine if they can deliver a sustained release of the drug to the solid tumor over a period of about two weeks. It has been shown, however, that while the microspheres could theoretically deliver a more prolonged dose of drug, the microspheres must first travel against a pressure gradient to reach the tumor core, due to the hypertension induced by the interstitial tumor fluid.

As demonstrated by Au et al, Cancer Research, (1998) 58(10):2141-8, however, drug penetration into the solid tumor, can be enhanced by apoptosis-inducing pre-treatment with paclitaxel.

Paclitaxel's cytotoxic and anti-tumor properties derive from its ability to promote apoptosis (programed cell death) by inducing the assembly of microtubules from tubulin dimers and preventing microtubules from depolymerization. The stabilized microtubules inhibit normal dynamic reorganization of the microtubule network that is essential for vital interphase and mitotic functions. In addition paclitaxel induces abnormal arrays or “bundles” of microtubules throughout the cell cycle and multiple asters of microtubules during mitosis.

Paclitaxel Formulations

Paclitaxel is substantially water insoluble and must be administered using a solubilizing carrier. The currently approved paclitaxel carrier formulation, marketed as TAXOL®, comprising paclitaxel dissolved in ethanol and CREMOPHOR® EL (polyoxyethylated castor oil).

The TAXOL® carrier CREMOPHOR® EL can cause side effects, such as anaphylaxis and severe hyper-sensitivity. (Sarosy and Reed, J Natl Med Assoc (1993) 85(6):427-31.) To reduce the side effects, current recommended treatment with TAXOL® includes pre-medication with corticosteroids, diphenhydramine and H₂ antagonists.

Several alternative carriers have been proposed to address the anaphylaxis and severe hyper-sensitivity caused by the CREMOPHOR® EL. For example, U.S. Pat. No. 5,684,169, which is incorporated by reference, discloses unbranched cyclodextrin or branched cyclodextrin inclusion complexes of paclitaxel which improves the solubility of paclitaxel in water. The complex is produced by adding an unbranched cyclodextrin or a branched cyclodextrin to paclitaxel at a molar ratio of 1-20 times with respect to paclitaxel. The cyclodextrin inclusion complex improves paclitaxel absorption in cancer patients by improving solubility.

U.S. Pat. No. 5,415,869, which is incorporated by reference, discloses paclitaxel or paclitaxel tumor-active analogs solubilized using one or more negatively charged phospholipids and one or more zwitterionic phospholipids. The phospholipid mixture entraps paclitaxel or the analog in a liposome. The liposome is in the form of particles having a size of 0.025 to 10 microns, with substantially no crystals of paclitaxel or the analog.

U.S. Pat. No. 5,580,575, which is incorporated by reference, discloses a therapeutic chemotherapeutic delivery system comprising gas-filled microspheres and a therapeutic chemotherapeutic, as well as, methods for employing such microspheres in therapeutic chemotherapeutic delivery. The preferred microspheres of the disclosure are gas-filled liposomes with an encapsulated chemotherapeutic. Methods of preparing such liposomes in chemotherapeutic delivery applications are also disclosed.

WO 99/13914, which is incorporated herein by reference, discloses that paclitaxel, and other slightly water soluble chemotherapeutics can be formulated without CREMOPHOR® EL or other toxic solubilizers by forming a water soluble homogeneous complex with plasma proteins, such as human serum albumin (HSA) or human gamma globulin (γ-globulin). As disclosed by WO 99/13914 homogeneous aqueous solutions up to at least 4.68 mM paclitaxel (4 mg/mL) can be formulated using HSA. The plasma proteins act as a slow release reservoir of paclitaxel. WO 99/13914 further discloses a dosage range of paclitaxel-HSA complex containing 70-280 mg of paclitaxel per treatment. Such formulations can be made bio-equivalent to the conventional CREMOPHOR® EL containing formulations.

Other formulations for administering paclitaxel are disclosed in U.S. Pat. Nos. 5,504,102 and 5,407,683, incorporated herein by reference.

In addition, the slow infusion of CREMOPHOR® EL solutions has been studied as a means of avoiding or ameliorating the side effects of the CREMOPHOR® EL vehicle. The most common dosage is 135-175 mg/m²CREMOPHOR® EL, which is administered over a 3 hour, 6 hour, or 24 hour dosage schedule. (See U.S. Pat. Nos. 5,641,803, and 5,621,001, both incorporated herein by reference.) Other dosing schedules have been suggested to reduce toxic side effects, including 96 hour infusion every 21 days (U.S. Pat. No. 5,496,846, incorporated herein by reference) and 60-180 minutes, repeated a plurality of times during a 21 day period, each infusion separated by an interval of between 4 to 5 days. (U.S. Pat. No. 5,696,153, which is incorporated herein by reference).

Paclitaxel Chemotherapy Reservoir

An alternative method of administering paclitaxel is using a chemotherapy reservoir. U.S. Pat. Nos. 5,846,565, 5,626,862 and 5,651,986, which are incorporated herein by reference, discloses a method and compositions for localized delivery of a chemotherapeutic agent to solid tumors, where the chemotherapeutic agent does not cross the blood-brain barrier and is characterized by poor bioavailability and/or short half-lives in vivo. The compositions consist of reservoirs which release the chemotherapeutic over an extended period while at the same time preserving the bio-activity and bio-availability of the agent. The preferred embodiment is a plurality of microspheres made from a biodegradable polymeric matrix. Alternatively reservoirs can be a plurality of microspheres made from a non-biodegradable polymers. In an alternative embodiment reservoirs may be or connected to implanted infusion pumps. The microspheres are implanted within or immediately adjacent to the tumors to be treated or the site where tumors have been surgically removed. The patents further disclose the efficacy of paclitaxel and camptothecin delivered in polymeric implants prepared by compression molding of biodegradable and non-biodegradable polymers, respectively.

U.S. Pat. No. 5,888,530, which is incorporated herein by reference, discloses a method of enhancing the amount of a pharmaceutical composition delivered to a target tissue site in a mammal, by creating a transient differential between the hydrostatic pressure in the target site and a region near the target tissue site. An apparatus for performing the method is provided. In one form that apparatus includes a pharmaceutical reservoir, pump, and an agent reservoir and pump.

Chemotherapy reservoirs are also disclosed in U.S. Pat. No. 5,470,311 which is incorporated herein by reference.

Initial results testing such chemotherapy reservoirs have been disappointing. While a significantly lowered side effect profile has been demonstrated, there are no indications of clinical improvement.

### SUMMARY OF THE INVENTION

The limitations of current chemotherapy reservoir technology may be due to the retention of the chemotherapeutic only on the tumor periphery or at the injection site due to the poor penetration and distribution of the chemotherapeutic as a result of the neoplasm's high interstitial fluid pressure. A more potent anti-tumor effect may be achieved by targeting the chemotherapy directly to the tumor, i.e., intratumorally, rather than by systemic infusion. It is theorized that the entry of microspheres to the solid tumor can be even further augmented if the initial drug injection administered to induce apoptosis is a more soluble form of Taxol, i.e., paclitaxel/HSA, a complex of Taxol and albumin, thereby increasing the apoptosis along further pressure gradients.

We now report delivery of an anti-cancer chemotherapeutic, such as paclitaxel, using a composition for local administration of an anti-tumor chemotherapeutic, as a chemotherapeutic reservoir to a patient having a tumor. This invention comprises a plurality of microspheres incorporating the anti-tumor chemotherapeutic; and, a suspending solution which surrounds the microspheres. Advantage is taken of plasma proteins, such as HSA, to act as a slow release reservoir for anti-cancer chemotherapeutic, such as paclitaxel.

The present invention provides a composition for administering an anti-tumor chemotherapeutic as a chemotherapeutic reservoir to a patient having a tumor, the composition comprising; a plurality of microspheres incorporating the anti-tumor chemotherapeutic; and, a suspending solution which surrounds the microspheres. The preferred embodiment is a plurality of microspheres made from a biodegradable polymeric matrix. Alternatively reservoirs can be a plurality of microspheres made from a non-biodegradable polymers.

The present invention provides also a method for administering an anti-tumor chemotherapeutic to a patient having a tumor, comprising the steps of delivering the anti-tumor chemotherapeutic as a chemotherapeutic reservoir to the tumor; and, releasing the anti-tumor chemotherapeutic from the chemotherapeutic reservoir to an interstitial space of the tumor in a therapeutically effective amount, wherein, the chemotherapeutic reservoir includes a plurality of microspheres incorporating the anti-tumor chemotherapeutic and a suspending solution which surrounds the microspheres.

### DETAILED DESCRIPTION OF THE INVENTION

A Composition for Administering an Anti-Tumor Chemotherapeutic as a Chemotherapeutic Reservoir

The present invention provides a composition for administering an anti-tumor chemotherapeutic as a chemotherapeutic reservoir to a patient having a tumor wherein the composition comprises a plurality of microspheres which incorporate the anti-tumor chemotherapeutic; and, a suspending solution which surrounds each microsphere. As used herein, the composition sometimes may be referred to as a device.

The preferred embodiment provides for a plurality of microspheres made from a biodegradable polymeric matrix. Alternatively reservoirs can be a plurality of microspheres made from a non-biodegradable polymers.

The anti-tumor chemotherapeutic is preferably in a formulation comprising a mixture of the anti-tumor chemotherapeutic and a plasma protein in an amount effective to solubilize the anti-tumor chemotherapeutic. Most preferably the plasma protein is selected from the group consisting of human serum albumin and γ-immunoglobulin. As disclosed by WO 99/13914, herein incorporated by reference, homogeneous aqueous solutions up to at least 4.68 mM paclitaxel (4 mg/mL) can be formulated using HSA. The plasma proteins act as a slow release reservoir of paclitaxel.

Methods for incorporating chemotherapeutics into a microspheres are disclosed in U.S. Pat. Nos. 5,684,169, 5,470,31 1, 5,580,575, 5,846,565, 5,626,862 and 5,651,986.

In one embodiment of the present invention, the anti-tumor chemotherapeutic may be contained within the microsphere. Optionally the anti-tumor chemotherapeutic may be attached to the microsphere. Attachment refers to attachment either inside or outside the microsphere.

In the present invention the longest diameter of the microspheres is preferably less than about 20 microns. The microspheres may be irregularly shaped. The microspheres as used herein also refers to microcapsules.

One embodiment of the present invention provides a plurality of microspheres made from a biodegradable polymeric matrix. The biodegradable polymer may be selected from the group consisting of polyacetic acid, polyglycolic acid and a co-polymer of polyglycolic and polyacetic acid.

In one embodiment, degradation of the biodegradable polymer releases the anti-tumor chemotherapeutic from the microspheres in a therapeutically effective amount. Preferably, up to about 50% of the anti-tumor chemotherapeutic is released from the microspheres within 24 hours after the administration of the microspheres to the patient. More preferably, between about 15 to about 25% of the anti-tumor chemotherapeutic is released from the microspheres within 24 hours after the administration of the microspheres to the patient.

Alternatively reservoirs can be a plurality of microspheres made from a non-biodegradable polymers. The non-biodegradable polymer is optionally ethylene-vinyl acetate copolymer.

The microspheres made from a biodegradable polymer or a non-biodegradable polymers may be constructed so that by slow diffusion the anti-tumor chemotherapeutic is released in a therapeutically effective amount over a period of time. Preferable the anti-tumor chemotherapeutic is released over a period of time lasting from 1 week to six months. Most preferably, the anti-tumor chemotherapeutic is released in a therapeutically effective amount over a period of time lasting from 3 weeks to 2 months.

The anti-tumor chemotherapeutic of the composition is preferably an apoptosis inducing chemotherapeutic. Preferably, the apoptosis inducing chemotherapeutic is paclitaxel. Alternatively, the apoptosis inducing chemotherapeutic is selected from the group consisting of cisplatin, adriamycin, butyric acid, cyclophosphamide, etoposide, amsacrine, genistein, and mitoguazone.

Preferably, the paclitaxel is at a concentration from about 0.1 to about 10 mg/mL. Most preferably the paclitaxel is at a concentration from about 0.5 to about 5 mg/mL.

The suspending solution of the composition may also comprise the anti-tumor chemotherapeutic. Preferably the suspending solution contains the formulation comprising a mixture of the anti-tumor chemotherapeutic and a plasma protein in an amount effective to solubilize the anti-tumor chemotherapeutic described for the plurality of microspheres above. Most preferably the plasma protein is selected from the group consisting of human serum albumin and γ-immunoglobulin.

In another embodiment, the plurality of microspheres and the suspending solution both contain paclitaxel. In this embodiment the paclitaxel in both the plurality of microspheres and in the solution is about 70 to about 280 mg. Preferably, the paclitaxel in both the plurality of microspheress and in the solution is at a concentration of about 135 mg/m² to about 175 mg/m².

In one preferred embodiment about 10% to about 90% of the paclitaxel is present in the plurality of microspheres. More preferably about 60% to about 90% of the paclitaxel is present in the plurality of microspheres. Most preferably, between about 80% to about 90% of the paclitaxel is present in the plurality of microspheres.

In an alternative embodiment, the suspending solution contains a second anti-tumor chemotherapeutic. The second anti-tumor chemotherapeutic is optionally an apoptosis inducing chemotherapeutic. The apoptosis inducing chemotherapeutic is selected from the group consisting of paclitaxel, cisplatin, adriamycin, butyric acid, cyclophosphamide, etoposide, amsacrine, genistein, and mitoguazone.

A Method for Delivering an Anti-Tumor Chemotherapeutic

The present invention also provides for a method for administering an anti-tumor chemotherapeutic to a patient having a tumor using the composition of the present invention. The method of administration comprises the steps of delivering the anti-tumor chemotherapeutic as a chemotherapeutic reservoir to the tumor; and, releasing the anti-tumor chemotherapeutic from the chemotherapeutic reservoir to an interstitial space of the tumor in a therapeutically effective amount, wherein, the chemotherapeutic reservoir includes a plurality of microspheres incorporating the anti-tumor chemotherapeutic and a suspending solution which surrounds the plurality of microspheres.

In one embodiment the delivering step includes the step of positioning chemotherapeutic reservoir within the tumor. The delivering step may include the step of intratumorally injecting the chemotherapeutic reservoir within the tumor. Alternatively, the delivering step includes the step of positioning chemotherapeutic reservoir adjacent to the tumor.

In one embodiment of the present invention composition is injected adjacent to the tumor or intra-tumorally using a syringe. Alternatively a syringe pump may be used to inject the composition. The flow rate and pressure of the syringe pump will depend upon the tumor to be treated. The flow rate of the syringe pump may vary from about 0.0167 mL/min to about 0.5 mL/min. The preferred flow rate will deliver the paclitaxel formulation to greater than 90% of the tumor volume while delivering essentially no paclitaxel outside the tumor.

In one embodiment, the releasing step includes the step of releasing the anti-tumor chemotherapeutic from the plurality of microspheres wherein degradation of the biodegradable polymer releases the anti-tumor chemotherapeutic from the microspheres in a therapeutically effective amount. A preferred releasing step includes releasing up to about 50% of the anti-tumor chemotherapeutic from the plurality of microspheres within 24 hours following delivery of the chemotherapeutic reservoir to the tumor. More preferably, the releasing step includes releasing between about 15 to about 25% of the anti-tumor chemotherapeutic from the plurality of microspheres within 24 hours following delivery of the chemotherapeutic reservoir to the tumor.

Alternatively, the reservoirs can be a plurality of microspheres made from a biodegradable or a non-biodegradable polymer. The non-biodegradable polymer is optionally ethylene-vinyl acetate copolymer.

The microspheres made from a biodegradable or a non-biodegradable polymers may be constructed so that by slow diffusion the anti-tumor chemotherapeutic is released in a therapeutically effective amount over a period of time. Preferable the anti-tumor chemotherapeutic is released over a period of time lasting from 1 week to six months. Most preferably, the anti-tumor chemotherapeutic is released in a therapeutically effective amount over a period of time lasting from 3 weeks to 2 months.

In one embodiment, the releasing step includes the step of diffusion of the anti-tumor chemotherapeutic to tumor cells as a soluble formulation. Optionally, the soluble formulation comprises a mixture of the anti-tumor chemotherapeutic and a plasma protein in an amount effective to solubilize the anti-tumor chemotherapeutic. Preferably, the plasma protein is selected from the group consisting of human serum albumin and γ-immunoglobulin. These plasma proteins facilitate defusion of the anti-tumor chemotherapeutic.

While not being bound by theory, it is proposed that administering a soluble form of an anti-tumor chemotherapeutic, such as a paclitaxel/plasma protein complex, increases drug efficacy by promoting paclitaxel diffusion. Increased diffusion promotes apoptosis tumor cell death not only in the immediate zone of the injection but also at sites further into the tumor where the paclitaxel has migrated.

The function and advantage of these and other embodiments of the present invention will be more fully understood from the examples below. The following examples are intended to illustrate the benefits of the present invention, but do not exemplify the full scope of the invention.

### EXAMPLES

### Example 1

### In Vivo Evaluation of the Effect of Paclitaxel/HSA to Disperse Microspheres within a Tumor in the Human Breast Tumor Xenograft (Cell line MCF7) in Nude Mice Study Objective

The purpose of the study is to compare the extent of the dispersal of fluorescently labeled microsphere particles injected into a solid tumor following an initial injection of Paclitaxel/HSA, relative to the dispersal of fluorescently labeled microspheres that is observed when no initial dose of Paclitaxel/HSA is administered.

Study Groups

There are five study groups consisting of 10 mice per group. The mice are allocated to the following 5 groups:

| | | Microspheres | |
|---|---|---|---|
| Group | Paclitaxel/HSA | with Flourescent | Number of |
| Number | Injection | Dye | Mice |
| I | − | + | 0 |
| II | + | + | 10 |
| III | − | + | 10 |
| | | (at elevated | |
| | | pressure) | |
| IV | + | + | 10 |
| | (at elevated | | |
| | pressure) | | |
| V | + | + | 10 |
| | (at elevated | (at elevated | |
| | pressure) | pressure) | |

Study Design

Immunodeficient nude (athymic mice) of approximately 5 weeks of age are injected subcutaneously with a cell suspension containing approximately 10⁷ cells/0. 1 ml of human mammary tumor cell line MCF7. The mice are examined routinely for the appearance of tumors. On Day 28 following tumor cell implantation, all tumors are measured as described below, and the measurement are recorded for each mouse as the “pre-treatment baseline tumor volume”. Tumor measurement are performed using calipers, to measure the tumor in two dimensions, at approximately 90° to each other, at the longest and widest points. The tumor volume will be calculated according to the formula, (W²×L)/2, where W is the tumor measurement at the widest point, and L is the tumor dimension at the longest point.

To ensure relative homogeneity of the tumor volumes, so that effective chemotherapeutic dispersal between groups can be compared, only mice with tumor volumes within the range of 5-8 grams are allocated to the study groups. At Day “0” of the Treatment Phase, Group I receive a reservoir injection of inert microspheres containing fluorescent dye only, while Group II receive an initial loading injection of Paclitaxel/HSA, followed within 24 hours by a second injection of inert microspheres containing fluorescent dye. Group III receive a reservoir injection of inert microspheres containing fluorescent dye only but delivered at elevated pressure. Group IV receive an initial loading injection of Paclitaxel/HSA delivered at elevated pressure, followed within 24 hours by a second injection of inert microspheres containing fluorescent dye delivered at regular pressure. Group V receive an initial loading injection of Paclitaxel/HSA delivered at elevated pressure, followed within 24 hours by a second injection of inert microspheres containing fluorescent dye delivered at elevated pressure. Within 24 hours after infusion of the microspheres in each group, the mice sacrificed and the tumors removed. Tumor tissues be fixed immediately and sectioned into 100 μm slices. The distribution area of fluorescent label in each slice are quantified using a macroimaging system, including a fluorescence stereo microscope equipped with a sensitive CCD camera. The distribution volume is calculated from the distribution area quantified in each slice.

Study Parameters

For each mouse within a study group, the distribution volume of the fluorescent dye within the microspheres injected are measured. The mean distribution volume for all mice within the group are determined and the values obtained for the two groups (microspheres alone versus microspheres following initial paclitaxel/HSA injection) are compared.

Results

The results of the distribution volume of the five groups are (expected results):

| Group number | Distribution volume (%) |
|---|---|
| I | 10 |
| II | 35 |
| III | 25 |
| IV | 45 |
| V | 80 |

Conclusions

Pre-injecting a soluble paclitaxel into the tumor causes apoptosis affording more efficient subsequent distribution of microspheres. Elevated pressure helps provide improved distribution in all cases. Elevated pressure for the pre-dose spreads the pre-dose to a larger portion of the tumor volume allowing the subsequent injection of the microspheres to spread. Elevated pressure for this injection too, results in a significant improvement in microsphere spread and has the potential of significantly improving the results of tumor shrinkage.

### Example 2

### In Vivo Evaluation of the Anti-Tumor Effect of an Intratumoral Injection of Paclitaxel Microspheres Suspended in Paclitaxel/HSA in Human Breast Tumor (Cell line MCF7) Xenografts in Nude Mice

Study Objective

The purpose of the study is to assess the anti-tumor effect of microspheres containing paclitaxel which are suspended in a solution of Paclitaxel/HSA (a novel proprietary compound of paclitaxel (Taxol) complexed with albumin) against a human mammary tumor xenograft (cell line MCF7) in immunodeficient mice. The potential of an intratumoral injection of the paclitaxel microsphere - Paclitaxel/HSA solution combination to reduce the xenograft tumor size are compared to the standard chemotherapeutic agent, Taxol.

Study Groups

There are five study groups containing 6-10 mice per group. The mice are allocated to the following 5 groups:

| Group | | | Method of | Number of Injections |
|---|---|---|---|---|
| Number | Chemotherapeutic | Dosage | Administration | (within 24 hours) |
| I | No treatment (control) | — | — | — |
| II | Saline (control) | 0.2.ml/gm^{a} | Intra-tumoral | 2 |
| III | Taxol | 0.2 ml/gm^{a} | Intra-tumoral | 2 |
| IV | Paclitaxel microspheres | 0.2 ml/gm^{a} | Intra-tumoral | 1 |
| | suspended in | | (via elevated | |
| | Paclitaxel/HSA | | pressure | |
| | | | infusion) | |
| V | Paclitaxel/HSA followed | 0.2 ml/gm^{b} | Intra-tumoral | 1 |
| | by paclitaxel | | (via elevated | |
| | microspheres suspended | 0.2 ml/gm^{a} | pressure | 1 |
| | in paclitaxel/HSA | | infusion) | |

^{a}per gram tumor weight at 60 mg paclitaxel/ml
^{b}per gram tumor weight at 1 mg paclitaxel/ml

Study Design

Nude (athymic mice) (˜5 weeks of age) are injected subcutaneously with a cell suspension containing approximately 10⁷ cells/0. 1 ml of human mammary tumor cell line MCF7. The mice are examined routinely for the appearance of tumors. On Day 28 following tumor cell implantation, all tumors are measured as described below, and the measurement recorded for each mouse as the pre-treatment baseline tumor volume. Tumor measurements are performed using calipers, to measure the tumor in two dimensions, at approximately 90° to each other, at the longest and widest points. The tumor volume are calculated according to the formula, (W²×L)/2, where W is the tumor measurement at the widest point, and L is the tumor dimension at the longest point.

All mice with tumor volumes within the range of 5-8 grams are allocated to study groups. Allocation to treatment groups are carried out based on the volume of the individual tumors, with each study group receiving an approximately equal representation of all tumor volumes. At study baseline, Day “0” of the Treatment Phase, all mice that are scheduled to receive two injections receive the first injection according to their study group assignment. Approximately twenty-three hours later, the tumors be measured as described above, and the volumes recorded. Immediately following measurement, within 24 hours of the first injection, the mice receive a second injection according to the study group assignment or their single injection. Post-treatment tumor volumes are assessed at 48 hours, 7 days, 14 days, and 21 days following the initial injection. The mice are sacrificed and the tumors removed and weighed. The final weights for each treatment group are averaged and compared to the final weights obtained for the “no-treatment” group.

Study Parameters

For each mouse within a study group, the post-treatment tumor volumes just before the 2^{nd} injection at 24 hours, and at 48 hours, 7, 14 and 21 days following the initial injection, are measured and recorded. The relative tumor volume (post-treatment tumor volume/pre-treatment baseline tumor volume) are recorded at each time point, and the mean relative tumor volume for each time point, for all mice within a study group, is determined. Additionally, following sacrifice, the final weights for the tumors for each study group are averaged and compared to the final weights observed for the “no-treatment” group.

Results

The results of relative tumor volume (100 x post-treatment tumor volume/pre-treatment baseline tumor volume) (expected results) are collected in the following table:

| | % volume | % volume | % volume | % volume |
|---|---|---|---|---|
| Group | 48 hour | 7 days | 14 days | 21 days |
| I | 105 | 125 | 150 | 175 |
| II | 105 | 125 | 150 | 150 |
| III | 70 | 70 | 100 | 130 |
| IV | 75 | 50 | 30 | 20 |
| V | 50 | 20 | 10 | 10 |

Conclusions

Infusion of paclitaxel microspheres suspended in a soluble paclitaxel intratumorally at elevated pressure allows spread of the microspheres to a large portion of the tumor volume. The extended release of the chemotherapeutic to a large percentage of the tumor volume, affords a significant tumor shrinkage. Pre-treatment with a soluble complex of paclitaxel about 24 hours before the infusion of the microsphere-soluble paclitaxel combination gives an improved efficacy in terms of tumor shrinkage.

What is claimed is:

1. A composition for local administration of an anti-tumor chemotherapeutic to a patient having a tumor, the composition comprising;
a plurality of microspheres incorporating the anti-tumor chemotherapeutic surrounded by a suspending solution.

2. The composition of claim 1, wherein the anti-tumor chemotherapeutic is in a formulation comprising a mixture of the anti-tumor chemotherapeutic and a plasma protein in an amount effective to solubilize the anti-tumor chemotherapeutic.

3. The composition of claim 2, wherein the plasma protein is selected from the group consisting of human serum albumin and γ-immunoglobulin.

4. The composition of claim 2, wherein the longest diameter of the microspheres is less than about 20 microns.

5. The composition of claim 2, wherein the microspheres are microcapsules.

6. The composition of claim 2, wherein the anti-tumor chemotherapeutic is contained within the microsphere.

7. The composition of claim 2, wherein the anti-tumor chemotherapeutic is attached to the microsphere.

8. The composition of claim 2, wherein the microspheres comprises a biodegradable polymer.

9. The composition of claim 8, wherein the biodegradable polymer is selected from the group consisting of polyacetic acid, polyglycolic acid and a co-polymer of polyglycolic and polyacetic acid.

10. The composition of claim 2, wherein the microspheres comprise a non-biodegradable polymer.

11. The composition of claim 13 wherein, the non-biodegradable polymer is an ethylene-vinyl acetate copolymer.

12. The composition of claim 2, wherein degradation of the microspheres releases the anti-tumor chemotherapeutic from the microspheres in a therapeutically effective amount.

13. The composition of claim 12, wherein up to about 50% of the anti-tumor chemotherapeutic is released from the microspheres within about 24 hours after administration of the microspheres to the patient.

14. The composition of claim 12, wherein between about 15 to about 25% of the anti-tumor chemotherapeutic is released from the microspheres within about 24 hours after administration of the microspheres to the patient.

15. The composition of claim 2, wherein the anti-tumor chemotherapeutic is released from the microsphere by diffusion.

16. The composition of claim 15, wherein the anti-tumor chemotherapeutic is released in a therapeutically effective amount over a period of time from about 1 week to about six months after administration to the patient.

17. The composition of claim 15, wherein the anti-tumor chemotherapeutic is released in a therapeutically effective amount over a period of time from about 3 weeks to about 2 months after administration to the patient.

18. The composition of claim 2, wherein the anti-tumor chemotherapeutic is an apoptosis inducing chemotherapeutic.

19. The composition of claim 18, wherein the apoptosis inducing chemotherapeutic is selected from the group consisting of cisplatin, adriamycin, butyric acid, cyclophosphamide, etoposide, amsacrine, genistein, and mitoguazone.

20. The composition of claim 18, wherein the anti-tumor chemotherapeutic is paclitaxel.

21. The composition of claim 20, wherein the paclitaxel is at a concentration from about 0.1 to about 10 mg/mL.

22. The composition of claim 20, wherein the paclitaxel is at a concentration from about 0.5 to about 5 mg/mL.

23. The composition of claim 2, wherein the suspending solution also includes an apoptosis inducing chemotherapeutic.

24. The composition of claim 23, wherein the apoptosis inducing chemotherapeutic is paclitaxel.

25. The composition of claim 24, wherein the paclitaxel in both the microspheres and in the solution is about 70 to about 280 mg.

26. The composition of claim 24, wherein the paclitaxel in both the microspheres and in the solution is at a concentration of about 135 mg/m² to about 175 mg/m².

27. The composition of claim 23, wherein between about 10% to about 90% of the paclitaxel is present in the microspheres.

28. The composition of claim 23, wherein between about 60% to about 90% of the paclitaxel is present in the microspheres.

29. The composition of claim 23, wherein between about 80% to about 90% of the paclitaxel is present in the microspheres.

30. The composition of claim 2, further comprising a second anti-tumor chemotherapeutic in the suspending solution.

31. The composition of claim 30, wherein the second anti-tumor chemotherapeutic is an apoptosis inducing chemotherapeutic.

32. The composition of claim 30, wherein the second anti-tumor chemotherapeutic is selected from the group consisting of paclitaxel, cisplatin, adriamycin, butyric acid, cyclophosphamide, etoposide, amsacrine, genistein, and mitoguazone.

33. A method for local administration of an anti-tumor chemotherapeutic to a tumor, comprising the steps of:
delivering a chemotherapeutic reservoir to the tumor; and,
releasing the chemotherapeutic from the reservoir to an interstitial space of the tumor in a therapeutically effective amount,
wherein, the chemotherapeutic reservoir includes a plurality of microspheres incorporating the anti-tumor chemotherapeutic and a suspending solution surrounding the microspheres.

34. The method of claim 33, wherein the chemotherapeutic reservoir comprises a mixture of the anti-tumor chemotherapeutic and a plasma protein in an amount effective to solubilize the anti-tumor chemotherapeutic.

35. The method of claim 34, wherein the plasma protein is selected from the group consisting of human serum albumin and γ-immunoglobulin.

36. The method of claim 34, wherein the microspheres comprise a biodegradable polymer.

37. The method of claim 36, wherein the biodegradable polymer is selected from the group consisting of polyacetic acid, polyglycolic acid and a co-polymer of polyglycolic and polyacetic acid.

38. The method of claim 34, wherein the microspheres comprise a non-biodegradable polymer.

39. The method of claim 38, wherein the non-biodegradable polymer is a ethylene-vinyl acetate copolymer.

40. The method of claim 34, wherein the anti-tumor agent is released from the microspheres in a therapeutically effective amount by degradation of the microspheres.

41. The method of claim 40, wherein about 50% of the anti-tumor chemotherapeutic from the microspheres within about 24 hours following delivery of the chemotherapeutic reservoir to the tumor.

42. The method of claim 40, wherein about 15 to about 25% of the anti-tumor chemotherapeutic from the microspheres within about 24 hours following delivery of the chemotherapeutic reservoir to the tumor.

43. The method of claim 34, wherein the anti-tumor chemotherapeutic is released from the microsphere by diffusion.

44. The method of claim 43, wherein the anti-tumor chemotherapeutic is continuously released from the microspheres in a therapeutically effective amount for a time period lasting from between about one week to about six months.

45. The method of claim 43, wherein the anti-tumor chemotherapeutic is continuously released from the microspheres in a therapeutically effective amount for a time period lasting from between about three weeks to about two months.

46. The method of claim 34, wherein the longest diameter of the microspheres are less than about 20 microns.

47. The method of claim 34, wherein the microspheres are microcapsules.

48. The method of claim 34, wherein the anti-tumor chemotherapeutic is an apoptosis inducing chemotherapeutic.

49. The method of claim 48, wherein the apoptosis inducing chemotherapeutic is selected from the group consisting of cisplatin, adriamycin, butyric acid, cyclophosphamide, etoposide, amsacrine, genistein, and mitoguazone.

50. The method of claim 48, wherein the anti-tumor chemotherapeutic is paclitaxel.

51. The composition of claim 50, wherein the paclitaxel is at a concentration from about 0.1 to about 10 mg/mL.

52. The method of claim 50, wherein the paclitaxel is at a concentration from about 0.5 to about 5 mg/mL.

53. The method of claim 34, wherein the suspending solution also includes an apoptosis inducing chemotherapeutic.

54. The method of claim 53, wherein the apoptosis inducing chemotherapeutic is paclitaxel.

55. The method of claim 53, wherein the total paclitaxel in both the microspheres and in the solution is about 70 to about 280 mg.

56. The method of claim 53, wherein the total paclitaxel in both the microspheres and in the solution is at a concentration of about 135 mg/m² to about 175 mg/m².

57. The method of claim 53, wherein between about 10% to about 90% of the paclitaxel is present in the microspheres.

58. The method of claim 53, wherein between about 60% to about 90% of the paclitaxel is present in the microspheres.

59. The method of claim 53, wherein between about 80% to about 90% of the paclitaxel is present in the microspheres.

60. The method of claim 34, further comprising a second anti-tumor chemotherapeutic in the suspending solution.

61. The method of claim 60, wherein the second anti-tumor chemotherapeutic is an apoptosis inducing chemotherapeutic.

62. The method of claim 60, wherein the second anti-tumor chemotherapeutic is selected from the group consisting of paclitaxel, cisplatin, adriamycin, butyric acid, cyclophosphamide, etoposide, amsacrine, genistein, and mitoguazone.

63. The method of claim 34, wherein the delivering step includes the step of positioning the chemotherapeutic reservoir within the tumor.

64. The method of claim 34, wherein the delivering step includes the step of intratumorally injecting the chemotherapeutic reservoir within the tumor.

65. The method of claim 34, wherein the delivering step includes the step of positioning chemotherapeutic reservoir adjacent to the tumor.
